(19) United States
(12) Patent Application Publication
(10) Pub. No.: US 2006/0116523 A1
(43) Pub. Date: Jun. 1, 2006

(54) Silver-containing catalysts for the reaction of substrates with c-c-double bonds

(76) Inventors: Wolfram Stichert, Ziegelhuasen (DE); Jens Klein, Heidelberg (DE); Stephan Andreas Schunk, Heidelberg (DE); Dirk Demuth, Walldorf (DE); Andreas Sundermann, Bensheim (DE); Ewald Gallei, Viernheim (DE); Harmut Hibst, Schriesheim (DE); Sebastian Storck, Mannheim (DE)

Correspondence Address: Stephen D Scanlon;Jones Day, North Point, 901 Lakeside Avenue, Cleveland, OH, 44114, US

(21) Appl. No.: 10/541,561

(22) PCT Filed: Jan. 9, 2004
(86) PCT No.: PCT/EP04/00112

§ 371 Date: Sep. 30, 2005

(30) Foreign Application Priority Data
Jan. 9, 2003 (DE) 103005269

(51) Int. Cl. C07D 301/10; B01J 23/50; B01J 23/48

(52) U.S. Cl. 549/534; 502/347

(57) ABSTRACT

The present invention relates to a silver-containing catalyst system, which can be applied for the reaction of substrates with at least one C—C-double bond with at least one oxygen-containing or oxygen-supplying component with formation of at least one epoxide. The silver-containing catalyst according to the invention is characterized in that its activity as well as its selectivity is significantly increased with respect to the target product compared with the silver-containing catalysts of the state of the art, as a consequence of the process for the manufacture according to the invention, in which the synthesis of a silver-amine complex is carried out in absence of light and at temperatures below room temperature.

The present invention relates to a silver-containing catalyst system, which can be applied for the reaction of substrates with at least one C—C-double bond with at least one oxygen-containing or oxygen-supplying component with formation of at least one epoxide. The silver-containing catalyst according to the invention is characterized in that its activity as well as its selectivity is significantly increased with respect to the target product compared with silver-containing catalysts of the state of the art, as a consequence of the process for the manufacture according to the invention, in which the synthesis of a silver-amine complex is carried out in absence of light and at temperatures below room temperature.

The use of silver-containing catalysts for reactions of substances with at least one C—C-double bond is discussed in detail in the state of the art.

So, the U.S. Pat. No. 2,279,470 describes in general the epoxidation of olefins in presence of molecular oxygen at “active silver surface catalysts”.

The EP-B 0 326 392 relates inter alia to the selective epoxidation of 1,3-butadiene to vinyl oxirane in presence of a silver-containing catalyst. Thereby, halogenated hydrocarbon is co-fed to the reaction in the ppm-range, the temperature range is restricted to 75° C.-325° C. as well as the olefin conversion is restricted to 0.1-75%. The respective US-patents of the patent family (for example U.S. Pat. No. 4,897,498, U.S. Pat. No. 4,950,773) are restricted to promoted silver catalysts, whereby alkali metal salts act as doping components.

In the U.S. Pat. No. 5,362,890, a saturated hydrocarbon is used additionally as co-feed. Said patent relates to a process for the manufacture of vinyl oxirane from, for example, 1,3-butadiene, whereby 40-90 mole-% of a paraffinic hydrocarbon is used in the educt feed as co-feed. Here, the range of the reaction temperature is between 175° C. and 230° C.

In none of the before-mentioned documents a silver-containing catalyst is disclosed, which was produced by means of controlled conditions, in particular the absence of light and at temperatures below standard room temperature (25° C.).

The object of the present invention was providing a silver-containing catalyst for the reaction of compounds with at least one C—C-double bond, in particular for the reaction of 1,3-buadiene to vinyl oxirane, which is characterized in an increased activity and/or conversion and/or selectivity compared with the state of the art when using similar reaction conditions.

Surprisingly, it was found, that clearly increased conversions, yields, and/or selectivities can be achieved, if the silver-containing catalyst is produced in absence of light and/or at temperatures below the room temperature.

The present invention relates to a silver-containing catalyst system, which can be applied for the reaction of substrates with at least one C—C-double bond with at least one oxygen-containing or oxygen-supplying component with formation of at least one epoxide. The silver-containing catalyst according to the invention is characterized in that its activity is significantly increased with respect to the silver-containing catalysts of the state of the art, as consequence of the process for the manufacture of the invention, in which the synthesis of a silver-amine complex is carried out in absence of light and at temperatures below the room temperature. Moreover, the present invention relates to the use of the addressed catalyst for the reaction of substrates with at least one C—C-double bond with at least one oxygen-containing or oxygen-supplying component with formation of at least one epoxide, in particular the corresponding reaction of 1,3-butadiene to vinyl oxirane.

An educt resp. intermediate, which is of particular importance for the chemical industry, is the vinyl oxirane (VO; 1,3-epoxybutene). VO is because of its double-functionality (reactive epoxide ring, double bond) an important (reactive) intermediate. For example, it can be rearranged to crotonaldehyde by means of a ring-opening isomerization, which, in turn, is an important intermediate in the synthesis of vitamin E, for the manufacture of sorbic acid (preservative in the food industry and animal feed industry) as well as for the synthesis of 3-methoxybutanol (lubricant, for example in shock absorbers). Also, the acid-catalyzed ring opening of the epoxide ring to the corresponding diol can be carried out easily.

Without restricting the general validity of the extent of protection scope of the present invention, the invention is exemplified in the following inter alia by means of the epoxidation of 1,3-butadiene to vinyl oxirane by using the silver-containing catalyst according to the invention. The reaction of 1,3-buadiene in the gas phase to vinyl oxirane is carried out according to the following reaction equation: 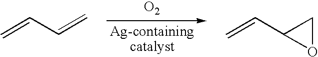
Reaction Equation:

Partial oxidation of 1,3-butadiene in presence of oxygen and in presence of a silver-containing catalyst system.

But, this does not deny that the catalyst according to the invention can also be applied for other reactions, containing the reaction of at least one C—C-double bond with at least one oxygen-containing and/or oxygen-supplying substance.

Essential terms, which are used in the present invention, shall be defined in the following:

An “epoxide” in the meaning of the present compound is any substance, which contains at least one oxygen atom, which has a bond to two vicinal carbon atoms, that means carbon atoms, which are linked by means of a chemical bond, which exceeds the degree of a physical interaction, that means which in particular is linked by means of a chemical (covalent) bond with said vicinal carbon atoms.

The terms “conversion”, “selectivity” and “yield”, which are used within the context of the present invention, are to be understood in that manner as defined in Fitzer, Fritz, Emig, Technische Chemie, Springer, Heidelberg, 4. Auflage, 1996.

The term “absence of light”, which is used within the context of the present invention, defines any condition, in which the access of light, that is of photons in the wavelength range of from 400 nm to 800 nm to the reaction space is reduced or prevented by means of constructive methods or other methods. There are no restrictions with respect to said methods.

The term “below reaction temperature”, which is used in the context of the present invention, defines any temperature, which is significantly below 25° C. in a manner that the reaction proceeds measurably different, for example slower, than said reaction would do at room temperature. In particular, the course of the side reaction of the reduction of silver ions to metallic silver is to be suppressed as far as possible.

Now, in the following, the invention shall be described in detail as well as preferred embodiments shall be specified.

There are no restrictions with respect to the manufacture of the catalysts, which are exemplified in the embodiments, apart from that the catalytic active material must contain at least silver according to the process for the manufacture, as well as that during the process for the manufacture at least partially absence of light and/or the existence of a temperature below room temperature is ensured.

Thereby, the silver can be supplied alone or in combination with at least one further element. Furthermore, it is preferred that the silver is applied at least partially onto at least one support. Thereby, the silver can be in metallic form, oxidic form, mixed-bonded form, as complexed ion, as reduced species as well as in a stoichiometric or in a non-stoichiometric composition.

Thereby, in a preferred embodiment, the silver is in complexed form. As complexing agent all substances can be used, the one skilled in the art knows from that said substances form with silver at least partially a coordination compound. Thereby, amines, diamines, alcohols, alkanediols, EDTA, functionalized carboxylic acid and carboxylic diacids are preferred. In particular, ethylenediamine is preferred. What is disclosed as aforesaid, preferably relates to all phases before the calcination.

In another preferred embodiment, the silver is in reduced form. As reducing agent, any substance, which is known to the one skilled in the art, can be applied in the synthesis of the active mass, which reduces at least partially the oxidation number of the silver in the respective condition on hand. It is preferred applying an alkane, an alcohol, an amine or another organic molecule, which by means of its redox potential is capable converting the material into a catalytic active and/or selective form. Thereby, the use of ethanolamine is in particular preferred.

In a particular preferred embodiment, the silver is both in reduced form and in complexed form. For example, such a process can be as follows: oxalic acid and ethylenediamine are charged. To it, silver oxide is added, which is dissolved in water. Ethanolamine is added to this mixture. Numerical values are specified in the embodiments. Now, said solution can be applied onto a support.

The active, silver-containing mass can be applied onto a support in any form, can brought into contact with a support or a support can be impregnated with said mass. Thereby, the silver resp. the silver-containing mass can be brought into contact with the support material out from the gas phase or from the liquid phase or as powder or in any combination of the before-mentioned processes.

The bringing into contact can consist of at least one of the processes of the group given below, however, without being restricted to said group: soaking, dunking, impregnating, deposition from the gas phase, mixing, grinding, sputtering, electrochemical deposition, chemical deposition without current, vacuum deposition, spreading of a paste-like mass, powder deposition, precipitation from or in a solution, spray drying. In particular, the application by means of bringing into contact of the support material with an aqueous phase is preferred.

In principle, as support material for the silver resp. the silver-containing mass all materials can be applied, which can be brought into contact with silver resp. the silver-containing mass. In a preferred embodiment, the at least one support material consists of at least one component selected from the following group: silicates, in particular SiO₂; alumina oxides, in particular α-Al₂O₃, γ-Al₂O₃; layer silicates, in particular steatite; oxides of the metals of the Main and Auxiliary Groups and, thereby, in particular TiO₂, ZrO₂; cerium oxide (oxides), mixed oxides, mixed oxides or oxides, in which parts of the lattice sites of a pure oxide, for example of a silicate, are replaced by at least one further element, and, thereby, in particular zeolites; carbon-containing supports and, thereby, in particular graphite and/or activated carbon, carbides; nitrides, as well as mixtures of at least two of the before-mentioned support materials. In the meaning of the present invention, Al₂O₃-containig supports are in particular preferred.

In the meaning of the present invention, the content of Ag with respect to the support material and expressed in weight-% ranges from 0.01% up to 10%. Thereby, in particular, a weight proportion of from 0.1% to 2.5% is preferred. Said weight proportion relates to the support steatite and is limited by a possible water absorption, which should not be incorporated in the above-mentioned numerical values.

The silver can form the catalyst as sole component or together with the support, or it is possible adding additional elements to the silver. Said additional elements can be elements from the Groups 1 to 17 of the Periodic Table of the Elements, and can preferably be selected from the Groups 1 to 12 and the Group 17. In particular preferred are the elements K, Rb, Cs, Sr and Ba. There is no restriction with respect to the number of additional elements and/or the proportion thereof.

In principle, in the present invention, the catalyst can be available as unsupported active mass (that means as full catalyst), or the catalyst can be available on one of the above-mentioned support materials (that means as shell catalyst, in case the support is not predominantly porous, or as support catalyst in case the support is predominantly porous).

In principle, the calcination of the catalyst, for example after the application of the silver resp. the silver-containing mass, and optional of an additional component and/or after a drying step, can take place at any temperature, which results among normal operating conditions in an economical tolerable durability of the catalyst for the catalytic application according to the invention. It is preferred applying to the calcination step temperatures between 200° C. and 800° C., and, temperatures of from 200° C. to 500° C. are in particular preferred, and further, temperatures of from 200° C. to below 300° C. are preferred.

The calcination can take place either in air or in a controlled atmosphere. Controlled atmospheres in the meaning of the present invention are: inert gases, reducing atmospheres, for example inert gases containing hydrogen, water steam, CO, CO₂, oxidizing atmospheres, reactive gases, atmospheres with increased or decreased pressure, in particular vacuum, as well as all possible combinations and/or mixtures of the before-mentioned atmospheres.

After the calcination, at least one step of the after-treatment can take place, whereby for the after-treatment in principle any step can be applied the one skilled in the art would apply for the after-treatment of catalysts in general.

In particular, the process for the manufacture of the catalyst according to the invention is characterized in that it is carried out in absence of light (as defined above) and/or at temperatures, which are diminished with respect to the room temperature (as likewise defined above). Said conditions must be fulfilled for at least one step of the manufacture of the catalyst, the calcination included. In a preferred embodiment, said condition or said conditions are fulfilled for all steps of the manufacture of the material according to the invention, the calcination being included.

Overall, the process for the manufacture of the silver-containing catalysts contains at least one of the two following steps:
(I) reaction of a silver-containing component with at least one complexing agent,
(II) reaction of a silver-containing component with at least one reducing agent,
   wherein at least one of the steps is carried out at least partially in absence of light or at temperatures below the room temperature or in absence of light and at temperatures below the room temperature.

Optionally, the process can contain at least one further of the following steps:
(III) bringing into contact of the silver-containing component from (I) and/or (II) with a support,
(IV) calcination of the support from (III), which was brought into contact,
   wherein also here one or both of said steps can be carried out optionally in absence of light or at temperatures below the room temperature, or in absence of light and at temperatures below the room temperature.

There are no restrictions with respect to the compounds, which have at least one C—C-double bond, and which are to be reacted using the silver-containing catalyst according to the invention. Thereby, it is preferred, using n-butene, like 1-butene and/or 2-butene (cis/trans). Thereby, the use of 1,3-butadiene is in particular preferred.

In principle, in the meaning of the present invention, there are no restrictions with respect to the oxygen-containing or oxygen-supplying components or substances, which are to be applied for the reaction with at least one compound, which contains at least one C—C-double bond. Thereby, oxygen, gases, which contain oxygen, in particular air, as well as water, aqueous mixtures, water steam, mixtures containing hydroperoxides in fluidic condition or any mixtures of at least two of the afore-mentioned substances are preferred. Furthermore, it is preferred that the oxygen-containing or oxygen-supplying components are predominantly in gaseous form, in particular if the reaction is to be carried out in a fixed bed.

In a preferred application, alkenes, preferably alkadienes, further preferred 1,3-butadiene are reacted in presence of oxygen or an oxygen-containing component of an educt gas to the corresponding epoxides, whereby the formation of vinyl oxirane from 1,3-butadiene is preferred, in the presence of the catalyst according to the invention, respectively.

In a preferred embodiment, the process for the reaction of the above-described educts in the gas phase in presence of one of the above-described catalysts with the target of the manufacture of epoxides, is carried out in at least one fixed bed reactor, which is charged with at least one of the silver-containing catalysts of the invention, whereby a tubular reactor with fixed bed is in particular preferred. By using the catalyst according to the invention, it is preferred using as reaction temperature according to the reaction of the invention a temperature between 225° C. and 350° C. For the space rate of the gas (GHSV) values of from 100 to 25.000 h⁻¹ are preferred, further preferred of from 2.000 to 20.000 h⁻¹. In the embodiments, conversion, selectivity and yield are shown for different temperatures and values of the GHSV.

The embodiments of the following should exemplify the present invention as well as their technical advantages. No restriction of the disclosure of the general description can be derived from the examples.

### EXAMPLE 1

### Reaction of Butadiene to Vinyl Oxirane by Using a Silver-Containing Catalyst T3131 (Comparison Example)

For the silver-containing catalyst system T3131, the synthesis of the silver-amine complex is carried out at room temperature and at daylight. Thereby, nitrates in aqueous solution are applied. For the synthesis of the silver-amine complex, 6.3 g oxalic acid, 6 g ethylenediamine and 11.6 g Ag₂O are dissolved in 25 ml H₂O and 1.7 g ethanolamine are added. Al₂O₃-beads from Ceramtec (granulate, diameter approximately 1 mm) are the support. In the present case, the silver load is 2.5 weight-% Ag on the support. Subsequently, the material is temperature-treated for 2 hours at 290° C. in the oven; thereby, the material is overflowed with 6 l/min air. In Table 1, exemplary results are given for the reaction of butadiene to vinyl oxirane (VO) by using the catalyst T3131. For this, for example 1 ml of the material (the catalyst volume is explicitly indicated in the tables) is inserted into a high-grade steel tubular reactor with an inner diameter of 8 mm (is inert among the reaction conditions, no activity with respect to the target reaction), and is heated from the outside up to the reaction temperature. The analysis of the product gas is carried out by means of a coupling of a micro-GC for the separation of the low boilers (butadiene) with a GC/MS with a Hewlett Packard HP-5 column for the separation and detection of the oxygenates.

TABLE 1

| Catalyst number. | mounting volume | temperature [° C.] | GHSV [h⁻¹] | convers. | sel. to VO | yield VO |
|---|---|---|---|---|---|---|
| T3131 | 2.0 ml | 180 | 12000 | 2.49 | 7.15 | 0.18 |
| T3131 | 1.0 mL | 180 | 12000 | 0.25 | 58.38 | 0.15 |
| T3131 | 1 ml/1 ml corundum | 180 | 12000 | 0.00 | 0.00 | 0.00 |
| T3131 | 1 ml/1 ml corundum | 180 | 6000 | 2.65 | 45.19 | 1.20 |
| T3131 | 1.0 mL | 180 | 6000 | 3.33 | 5.73 | 0.19 |
| T3131 | 2.0 ml | 180 | 6000 | 1.96 | 0.00 | 0.00 |
| T3131 | 1 ml/1 ml corundum | 200 | 12000 | 3.72 | 100.00 | 3.72 |
| T3131 | 1.0 mL | 200 | 12000 | 0.00 | 0.00 | 0.00 |
| T3131 | 2.0 ml | 200 | 12000 | 0.00 | 0.00 | 0.00 |
| T3131 | 1 ml/1 ml corundum | 200 | 6000 | 6.37 | 100.00 | 6.37 |
| T3131 | 2.0 ml | 200 | 6000 | 0.35 | 100.00 | 0.35 |
| T3131 | 1.0 mL | 200 | 6000 | 0.00 | 0.00 | 0.00 |
| T3131 | 1 ml/1 ml corundum | 200 | 3000 | 5.81 | 100.00 | 5.81 |
| T3131 | 1.0 mL | 200 | 3000 | 1.34 | 79.55 | 1.07 |
| T3131 | 2.0 ml | 200 | 3000 | 3.46 | 6.34 | 0.22 |
| T3131 | 1 ml/1 ml corundum | 220 | 12000 | 1.91 | 100.00 | 1.91 |
| T3131 | 1.0 mL | 220 | 12000 | 1.84 | 29.58 | 0.54 |
| T3131 | 2.0 ml | 220 | 12000 | 0.48 | 0.00 | 0.00 |
| T3131 | 1 ml/1 ml corundum | 220 | 6000 | 11.51 | 100.00 | 11.51 |
| T3131 | 2.0 ml | 220 | 6000 | 2.09 | 67.08 | 1.40 |
| T3131 | 1.0 mL | 220 | 6000 | 0.74 | 100.00 | 0.74 |
| T3131 | 1 ml/1 ml corundum | 220 | 3000 | 23.85 | 100.00 | 23.85 |
| T3131 | 1.0 mL | 220 | 3000 | 4.20 | 97.57 | 4.09 |
| T3131 | 2.0 ml | 220 | 3000 | 4.16 | 41.65 | 1.73 |
| T3131 | 1 ml/1 ml corundum | 240 | 12000 | 13.29 | 100.00 | 13.29 |
| T3131 | 1.0 mL | 240 | 12000 | 4.91 | 99.04 | 4.86 |
| T3131 | 2.0 ml | 240 | 12000 | 5.65 | 82.20 | 4.64 |
| T3131 | 1 ml/1 ml corundum | 240 | 6000 | 22.26 | 100.00 | 22.26 |
| T3131 | 2.0 ml | 240 | 6000 | 5.35 | 100.00 | 5.35 |
| T3131 | 1.0 mL | 240 | 6000 | 3.80 | 100.00 | 3.80 |
| T3131 | 1 ml/1 ml corundum | 240 | 3000 | 32.13 | 100.00 | 32.13 |
| T3131 | 1.0 mL | 240 | 3000 | 8.40 | 100.00 | 8.40 |
| T3131 | 2.0 ml | 240 | 3000 | 4.78 | 100.00 | 4.78 |
| T3131 | 1 ml/1 ml corundum | 260 | 12000 | 32.53 | 100.00 | 32.53 |
| T3131 | 1.0 mL | 260 | 12000 | 19.17 | 100.00 | 19.17 |
| T3131 | 2.0 ml | 260 | 12000 | 21.14 | 80.53 | 17.02 |
| T3131 | 1 ml/1 ml corundum | 260 | 6000 | 40.64 | 91.95 | 37.37 |
| T3131 | 2.0 ml | 260 | 6000 | 13.30 | 96.49 | 12.83 |
| T3131 | 1.0 mL | 260 | 6000 | 11.12 | 100.00 | 11.12 |
| T3131 | 1.0 mL | 260 | 3000 | 23.46 | 70.50 | 16.54 |
| T3131 | 1 ml/1 ml corundum | 260 | 3000 | 68.38 | 16.84 | 11.52 |
| T3131 | 2.0 ml | 260 | 3000 | 18.10 | 42.84 | 7.76 |
| T3131 | 1.0 mL | 280 | 12000 | 46.19 | 53.75 | 24.83 |
| T3131 | 2.0 ml | 280 | 12000 | 56.48 | 26.40 | 14.91 |
| T3131 | 1 ml/1 ml corundum | 280 | 12000 | 79.47 | 7.83 | 6.23 |
| T3131 | 1.0 mL | 280 | 6000 | 30.49 | 67.81 | 20.68 |
| T3131 | 2.0 ml | 280 | 6000 | 36.32 | 30.23 | 10.98 |
| T3131 | 1 ml/1 ml corundum | 280 | 6000 | 88.05 | 0.00 | 0.00 |
| T3131 | 1.0 mL | 280 | 3000 | 62.69 | 9.88 | 6.19 |
| T3131 | 1 ml/1 ml corundum | 280 | 3000 | 89.55 | 0.00 | 0.00 |
| T3131 | 2.0 ml | 280 | 3000 | 95.38 | 0.00 | 0.00 |
| T3131 | 1.0 mL | 300 | 12000 | 93.61 | 0.00 | 0.00 |
| T3131 | 1 ml/1 ml corundum | 300 | 12000 | 88.26 | 0.00 | 0.00 |
| T3131 | 2.0 ml | 300 | 12000 | 100.00 | 0.00 | 0.00 |
| T3131 | 1.0 mL | 300 | 6000 | 92.52 | 0.00 | 0.00 |
| T3131 | 1 ml/1 ml corundum | 300 | 6000 | 87.39 | 0.00 | 0.00 |
| T3131 | 2.0 ml | 300 | 6000 | 100.00 | 0.00 | 0.00 |
| T3131 | 1.0 mL | 320 | 12000 | 93.85 | 0.00 | 0.00 |
| T3131 | 1 ml/1 ml corundum | 320 | 12000 | 89.07 | 0.00 | 0.00 |
| T3131 | 2.0 ml | 320 | 12000 | 100.00 | 0.00 | 0.00 |

### EXAMPLE 2

### Reaction of Butadiene to Vinyl Oxirane by Using the Silver-Containing Catalyst T3326 of the Invention

For the silver-containing catalyst system T3326, the synthesis is carried out analogously to Example 1. The only differences are that here the synthesis is carried out in darkness and by means of cooling with an ice bath (0° C.). Furthermore, 0.001 weight-% Cs are added in form of CsNO₃ as doping component. Exemplary results are indicated in Table 2 for the reaction of butadiene to vinyl oxirane (VO) by using the catalyst T3326.

Compared with the catalyst from Example 1, which was not subjected to the process for the manufacture according to the invention, it can clearly be realized that yields can be achieved with respect to the vinyl oxirane, which are better up to a factor 3 (with the same or improved selectivity). In particular, this applies in the meaning that yield, conversion and selectivity are improved over the complete breadth of the conditions, that means that yield, conversion and selectivity are improved systematically. Thus, the technical advantage of the silver-containing catalyst according to the invention can clearly be realized for the one skilled in the art.

TABLE 2

| catalyst number | mounting volume | temperature [° C.] | GHSV [h⁻¹] | convers. | sel. to VO | yield VO |
|---|---|---|---|---|---|---|
| T3326 | 2.0 ml | 180 | 12000 | 15.49 | 100.00 | 15.49 |
| T3326 | 1 ml/1 ml corundum | 180 | 12000 | 10.11 | 100.00 | 10.11 |
| T3326 | 1.0 mL | 180 | 12000 | 7.87 | 100.00 | 7.87 |
| T3326 | 2.0 ml | 180 | 6000 | 22.95 | 100.00 | 22.95 |
| T3326 | 1 ml/1 ml corundum | 180 | 6000 | 20.24 | 100.00 | 20.24 |
| T3326 | 1.0 mL | 180 | 6000 | 15.60 | 100.00 | 15.60 |
| T3326 | 2.0 ml | 200 | 12000 | 36.48 | 100.00 | 36.48 |
| T3326 | 1 ml/1 ml corundum | 200 | 12000 | 26.59 | 100.00 | 26.59 |
| T3326 | 1.0 mL | 200 | 12000 | 20.84 | 100.00 | 20.84 |
| T3326 | 2.0 ml | 200 | 6000 | 46.15 | 100.00 | 46.15 |
| T3326 | 1 ml/1 ml corundum | 200 | 6000 | 38.78 | 100.00 | 38.78 |
| T3326 | 1.0 mL | 200 | 6000 | 32.09 | 100.00 | 32.09 |
| T3326 | 2.0 ml | 200 | 3000 | 63.69 | 100.00 | 63.69 |
| T3326 | 1 ml/1 ml corundum | 200 | 3000 | 50.20 | 100.00 | 50.20 |
| T3326 | 1.0 mL | 200 | 3000 | 49.71 | 100.00 | 49.71 |
| T3326 | 2.0 ml | 220 | 12000 | 56.54 | 100.00 | 56.54 |
| T3326 | 1 ml/1 ml corundum | 220 | 12000 | 43.54 | 100.00 | 43.54 |
| T3326 | 1.0 mL | 220 | 12000 | 43.01 | 100.00 | 43.01 |
| T3326 | 2.0 ml | 220 | 6000 | 67.48 | 100.00 | 67.48 |
| T3326 | 1 ml/1 ml corundum | 220 | 6000 | 56.93 | 100.00 | 56.93 |
| T3326 | 1.0 mL | 220 | 6000 | 54.81 | 100.00 | 54.81 |
| T3326 | 2.0 ml | 220 | 3000 | 83.25 | 100.00 | 83.25 |
| T3326 | 1.0 mL | 220 | 3000 | 74.78 | 100.00 | 74.78 |
| T3326 | 1 ml/1 ml corundum | 220 | 3000 | 71.19 | 100.00 | 71.19 |
| T3326 | 2.0 ml | 240 | 12000 | 82.33 | 100.00 | 82.33 |
| T3326 | 1.0 mL | 240 | 12000 | 66.61 | 100.00 | 66.61 |
| T3326 | 1 ml/1 ml corundum | 240 | 12000 | 61.88 | 100.00 | 61.88 |
| T3326 | 2.0 ml | 240 | 6000 | 81.26 | 100.00 | 81.26 |
| T3326 | 1.0 mL | 240 | 6000 | 72.71 | 100.00 | 72.71 |
| T3326 | 1 ml/1 ml corundum | 240 | 6000 | 69.91 | 100.00 | 69.91 |
| T3326 | 1.0 mL | 240 | 3000 | 86.89 | 100.00 | 86.89 |
| T3326 | 2.0 ml | 240 | 3000 | 89.99 | 81.80 | 73.62 |
| T3326 | 1 ml/1 ml corundum | 240 | 3000 | 79.50 | 77.05 | 61.25 |
| T3326 | 2.0 ml | 260 | 12000 | 89.95 | 84.07 | 75.62 |
| T3326 | 1 ml/1 ml corundum | 260 | 12000 | 70.99 | 100.00 | 70.99 |
| T3326 | 1.0 mL | 260 | 12000 | 97.12 | 10.39 | 10.09 |
| T3326 | 1.0 mL | 260 | 6000 | 86.65 | 100.00 | 86.65 |
| T3326 | 2.0 ml | 260 | 6000 | 90.62 | 75.77 | 68.66 |
| T3326 | 1 ml/1 ml corundum | 260 | 6000 | 77.06 | 87.20 | 67.20 |
| T3326 | 1.0 mL | 260 | 3000 | 95.49 | 41.38 | 39.51 |
| T3326 | 1 ml/1 ml corundum | 260 | 3000 | 82.87 | 27.89 | 23.11 |
| T3326 | 2.0 ml | 260 | 3000 | 93.21 | 12.76 | 11.89 |
| T3326 | 1 ml/1 ml corundum | 280 | 12000 | 76.05 | 60.61 | 46.10 |
| T3326 | 2.0 ml | 280 | 12000 | 100.00 | 4.66 | 4.66 |
| T3326 | 1.0 mL | 280 | 12000 | 100.00 | 2.23 | 2.23 |
| T3326 | 1 ml/1 ml corundum | 280 | 6000 | 81.11 | 37.78 | 30.64 |
| T3326 | 1.0 mL | 280 | 6000 | 95.32 | 12.47 | 11.89 |
| T3326 | 2.0 ml | 280 | 6000 | 95.17 | 1.63 | 1.55 |
| T3326 | 1 ml/1 ml corundum | 280 | 3000 | 85.00 | 3.33 | 2.83 |
| T3326 | 1.0 mL | 280 | 3000 | 100.00 | 1.35 | 1.35 |
| T3326 | 2.0 ml | 280 | 3000 | 91.35 | 0.00 | 0.00 |
| T3326 | 1 ml/1 ml corundum | 300 | 12000 | 79.43 | 28.68 | 22.78 |
| T3326 | 2.0 ml | 300 | 12000 | 100.00 | 0.86 | 0.86 |
| T3326 | 1.0 mL | 300 | 12000 | 100.00 | 0.57 | 0.57 |
| T3326 | 1 ml/1 ml corundum | 300 | 6000 | 85.26 | 9.40 | 8.01 |
| T3326 | 1.0 mL | 300 | 6000 | 100.00 | 0.98 | 0.98 |
| T3326 | 2.0 ml | 300 | 6000 | 100.00 | 0.00 | 0.00 |
| T3326 | 1 ml/1 ml corundum | 320 | 12000 | 80.88 | 11.72 | 9.48 |
| T3326 | 1.0 mL | 320 | 12000 | 100.00 | 0.26 | 0.26 |
| T3326 | 2.0 ml | 320 | 12000 | 100.00 | 0.16 | 0.16 |

### EXAMPLE 3

### Reaction of Butadiene to Vinyl Oxirane by Using the Silver-Containing Catalyst T3327 According to the Invention

The synthesis of the silver-containing catalyst system T3327 is carried out analogously to Example 1. The only differences to Example 1 are here the lead-through of the synthesis in darkness and by means of cooling with an ice bath 0° C.). Here, in difference to Example 2, an undoped Ag-catalyst is used. Exemplary results are indicated in Table 3 for the reaction of butadiene to vinyl oxirane (VO) by using the catalyst T3327. Thereby, the conversions, selectivities and yields are not as good as in Example 2, but better than in Example 1.

TABLE 3

| catalyst number | mounting volume | temperature [° C.] | GHSV [h⁻¹] | convers. | sel.to VO | yield VO |
|---|---|---|---|---|---|---|
| T3327 | 2.0 ml | 180 | 12000 | 1.20 | 49.43 | 0.59 |
| T3327 | 1 ml/1 ml corundum | 180 | 12000 | 0.53 | 100.00 | 0.53 |
| T3327 | 1.0 mL | 180 | 12000 | 0.55 | 65.28 | 0.36 |
| T3327 | 1 ml/1 ml corundum | 200 | 12000 | 3.85 | 100.00 | 3.85 |
| T3327 | 2.0 ml | 200 | 12000 | 0.55 | 100.00 | 0.55 |
| T3327 | 1.0 mL | 200 | 12000 | 0.00 | 0.00 | 0.00 |
| T3327 | 1 ml/1 ml corundum | 220 | 12000 | 4.35 | 77.36 | 3.36 |
| T3327 | 1.0 mL | 220 | 12000 | 3.31 | 46.58 | 1.54 |
| T3327 | 2.0 ml | 220 | 12000 | 2.71 | 55.34 | 1.50 |
| T3327 | 1 ml/1 ml corundum | 240 | 12000 | 16.77 | 100.00 | 16.77 |
| T3327 | 2.0 ml | 240 | 12000 | 13.45 | 100.00 | 13.45 |
| T3327 | 1.0 mL | 240 | 12000 | 9.88 | 100.00 | 9.88 |
| T3327 | 1 ml/1 ml corundum | 260 | 12000 | 40.09 | 94.49 | 37.88 |
| T3327 | 1.0 mL | 260 | 12000 | 36.40 | 100.00 | 36.40 |
| T3327 | 2.0 ml | 260 | 12000 | 62.01 | 54.96 | 34.08 |
| T3327 | 2.0 ml | 280 | 12000 | 100.00 | 0.00 | 0.00 |
| T3327 | 1.0 mL | 280 | 12000 | 98.31 | 0.00 | 0.00 |
| T3327 | 1 ml/1 ml corundum | 280 | 12000 | 84.68 | 0.00 | 0.00 |
| T3327 | 2.0 ml | 300 | 12000 | 100.00 | 0.00 | 0.00 |
| T3327 | 1.0 mL | 300 | 12000 | 100.00 | 0.00 | 0.00 |
| T3327 | 1 ml/1 ml corundum | 300 | 12000 | 86.03 | 0.00 | 0.00 |
| T3327 | 2.0 ml | 320 | 12000 | 100.00 | 0.00 | 0.00 |
| T3327 | 1.0 mL | 320 | 12000 | 100.00 | 0.00 | 0.00 |
| T3327 | 1 ml/1 ml corundum | 320 | 12000 | 83.26 | 0.00 | 0.00 |

### EXAMPLE 4

### Reaction of Butadiene to Vinyl Oxirane by Using the Silver-Containing Catalyst T3321 According to the Invention

The synthesis for the silver-containing catalyst system T3321 is carried out analogously to Example 2, however at room temperature. Exemplary results are indicated in Table 4 for the reaction of butadiene to vinyl oxirane (VO) by using the catalyst T3321. Here, too, conversions, selectivities and yields are clearly better than in Example 1 (Comparison Example), which represents the state of the art. Therewith, it is shown that already the absence of light leads to an essential improvement of the catalyst properties.

TABLE 4

| catalyst number | mounting volume | temperature [° C.] | GHSV [h⁻¹] | convers. | sel. to VO | yield VO |
|---|---|---|---|---|---|---|
| T3321 | 2.0 ml | 180 | 12000 | 15.28 | 100.00 | 15.28 |
| T3321 | 1.0 mL | 180 | 12000 | 8.70 | 100.00 | 8.70 |
| T3321 | 1 ml/1 ml corundum | 180 | 12000 | 6.82 | 100.00 | 6.82 |
| T3321 | 2.0 ml | 180 | 6000 | 18.31 | 100.00 | 18.31 |
| T3321 | 1.0 mL | 180 | 6000 | 15.37 | 100.00 | 15.37 |
| T3321 | 1 ml/1 ml corundum | 180 | 6000 | 14.88 | 100.00 | 14.88 |
| T3321 | 2.0 ml | 200 | 12000 | 30.23 | 100.00 | 30.23 |
| T3321 | 1.0 mL | 200 | 12000 | 21.63 | 100.00 | 21.63 |
| T3321 | 1 ml/1 ml corundum | 200 | 12000 | 19.87 | 100.00 | 19.87 |
| T3321 | 2.0 ml | 200 | 6000 | 41.04 | 100.00 | 41.04 |
| T3321 | 1.0 mL | 200 | 6000 | 30.64 | 100.00 | 30.64 |
| T3321 | 1 ml/1 ml corundum | 200 | 6000 | 30.46 | 100.00 | 30.46 |
| T3321 | 2.0 ml | 200 | 3000 | 63.38 | 100.00 | 63.38 |
| T3321 | 1.0 mL | 200 | 3000 | 50.29 | 100.00 | 50.29 |
| T3321 | 1 ml/1 ml corundum | 200 | 3000 | 38.76 | 100.00 | 38.76 |
| T3321 | 2.0 ml | 220 | 12000 | 37.71 | 100.00 | 37.71 |
| T3321 | 1 ml/1 ml corundum | 220 | 12000 | 36.65 | 100.00 | 36.65 |
| T3321 | 1.0 mL | 220 | 12000 | 33.22 | 100.00 | 33.22 |
| T3321 | 2.0 ml | 220 | 6000 | 67.10 | 100.00 | 67.10 |
| T3321 | 1.0 mL | 220 | 6000 | 54.69 | 100.00 | 54.69 |
| T3321 | 1 ml/1 ml corundum | 220 | 6000 | 44.74 | 100.00 | 44.74 |
| T3321 | 2.0 ml | 220 | 3000 | 84.33 | 100.00 | 84.33 |
| T3321 | 1.0 mL | 220 | 3000 | 72.58 | 100.00 | 72.58 |
| T3321 | 1 ml/1 ml corundum | 220 | 3000 | 55.90 | 100.00 | 55.90 |
| T3321 | 2.0 ml | 240 | 12000 | 81.72 | 100.00 | 81.72 |
| T3321 | 1.0 mL | 240 | 12000 | 63.68 | 100.00 | 63.68 |
| T3321 | 1 ml/1 ml corundum | 240 | 12000 | 58.98 | 100.00 | 58.98 |
| T3321 | 2.0 ml | 240 | 6000 | 84.27 | 100.00 | 84.27 |
| T3321 | 1.0 mL | 240 | 6000 | 72.67 | 100.00 | 72.67 |
| T3321 | 1 ml/1 ml corundum | 240 | 6000 | 58.97 | 100.00 | 58.97 |
| T3321 | 1.0 mL | 240 | 3000 | 86.85 | 90.60 | 78.68 |
| T3321 | 1 ml/1 ml corundum | 240 | 3000 | 68.43 | 74.38 | 50.89 |
| T3321 | 2.0 ml | 240 | 3000 | 95.59 | 50.79 | 48.55 |
| T3321 | 1.0 mL | 260 | 12000 | 78.43 | 100.00 | 78.43 |
| T3321 | 2.0 ml | 260 | 12000 | 93.19 | 72.63 | 67.68 |
| T3321 | 1 ml/1 ml corundum | 260 | 12000 | 68.09 | 89.97 | 61.26 |
| T3321 | 1.0 mL | 260 | 6000 | 85.72 | 89.77 | 76.96 |
| T3321 | 1 ml/1 ml corundum | 260 | 6000 | 67.56 | 85.79 | 57.96 |
| T3321 | 2.0 ml | 260 | 6000 | 94.43 | 60.67 | 57.30 |
| T3321 | 1 ml/1 ml corundum | 260 | 3000 | 74.19 | 29.00 | 21.51 |
| T3321 | 1.0 mL | 260 | 3000 | 93.90 | 21.16 | 19.87 |
| T3321 | 2.0 ml | 260 | 3000 | 100.00 | 3.77 | 3.77 |
| T3321 | 1 ml/1 ml corundum | 280 | 12000 | 73.59 | 43.89 | 32.30 |
| T3321 | 1.0 mL | 280 | 12000 | 94.18 | 24.19 | 22.78 |
| T3321 | 2.0 ml | 280 | 12000 | 100.00 | 10.12 | 10.12 |
| T3321 | 1 ml/1 ml corundum | 280 | 6000 | 72.50 | 38.33 | 27.79 |
| T3321 | 1.0 mL | 280 | 6000 | 91.96 | 9.99 | 9.19 |
| T3321 | 2.0 ml | 280 | 6000 | 100.00 | 4.56 | 4.56 |
| T3321 | 1 ml/1 ml corundum | 280 | 3000 | 76.78 | 4.34 | 3.33 |
| T3321 | 1.0 mL | 280 | 3000 | 93.99 | 0.41 | 0.38 |
| T3321 | 2.0 ml | 280 | 3000 | 100.00 | 0.00 | 0.00 |
| T3321 | 1 ml/1 ml corundum | 300 | 12000 | 75.53 | 25.26 | 19.08 |
| T3321 | 1.0 mL | 300 | 12000 | 96.11 | 5.24 | 5.03 |
| T3321 | 2.0 ml | 300 | 12000 | 100.00 | 1.12 | 1.12 |
| T3321 | 1 ml/1 ml corundum | 300 | 6000 | 73.66 | 10.66 | 7.85 |
| T3321 | 1.0 mL | 300 | 6000 | 93.20 | 0.50 | 0.46 |
| T3321 | 2.0 ml | 300 | 6000 | 100.00 | 0.12 | 0.12 |
| T3321 | 1 ml/1 ml corundum | 320 | 12000 | 76.41 | 10.27 | 7.85 |
| T3321 | 1.0 mL | 320 | 12000 | 96.45 | 1.67 | 1.61 |
| T3321 | 2.0 ml | 320 | 12000 | 100.00 | 0.24 | 0.24 |

### EXAMPLE 5

### Reaction of Butadiene to Vinyl Oxirane by Using a Silver-Containing Catalyst T2502 (Lower Silver Content: Comparison Example)

For the manufacture of the silver-containing catalyst system T2502, the synthesis of the silver-amine complex was carried out at room temperature and at daylight. Thereby, the nitrates are applied in aqueous solution. For the synthesis of the silver-amine complex 2.52 g oxalic acid, 2.4 g ethylenediamine and 4.635 g Ag₂O are dissolved in 10 ml H₂O , and 1.36 g ethanolamine are added. Al₂O₃-beads from Ceramtec are the support. In the present case, the load with silver is 0.5 weight-% Ag on the support. Subsequently, the material is temperature-treated for 3 hours at 290° C. in the oven; thereby, the material is overflowed with 6 l/min air. In Table 5, exemplary results are summarized for the reaction of butadiene to vinyl oxirane by using the catalyst T2502. The testing takes place analogously to Example 1.

TABLE 5

| catalyst number | mounting volume | temperature [° C.] | GHSV [h⁻¹] | convers. | sel. to VO | yield VO |
|---|---|---|---|---|---|---|
| T2502 | 1.0 mL | 220 | 12000 | 0.00 | 0.00 | 0.00 |
| T2502 | 1.0 mL | 240 | 12000 | 2.86 | 38.09 | 1.09 |
| T2502 | 1.0 mL | 260 | 12000 | 9.60 | 100.00 | 9.60 |
| T2502 | 1.0 mL | 280 | 12000 | 26.77 | 100.00 | 26.77 |
| T2502 | 1.0 mL | 300 | 12000 | 63.48 | 55.53 | 35.25 |
| T2502 | 1.0 mL | 320 | 12000 | 100.00 | 0.15 | 0.15 |
| T2502 | 1.0 mL | 340 | 12000 | 100.00 | 0.00 | 0.00 |
| T2502 | 1.0 mL | 340 | 12000 | 100.00 | 0.00 | 0.00 |
| T2502 | 1.0 mL | 360 | 12000 | 100.00 | 0.00 | 0.00 |
| T2502 | 1.0 mL | 380 | 12000 | 100.00 | 0.00 | 0.00 |

### EXAMPLE 6

### Reaction of Butadiene to Vinyl Oxirane by Using a Silver-Containing Catalyst T2530 (Comparison Example)

For the synthesis of the silver-containing catalyst system T2530, the synthesis of the silver-amine complex was carried out at room temperature and at daylight. Thereby, the nitrates are applied in aqueous solution. For the synthesis of the Ag-amine complex 2.52 g oxalic acid, 1.2 g ethylenediamine and 4.635 g Ag₂O are dissolved in 10 ml H₂O, and 0.68 g ethanolamine are added. Al₂O₃-beads from Ceramtec are the support. In the present case, the silver load is 1.0 weight-% Ag on the support. Subsequently, the material is temperature-treated for 3 hours at 290° C. in the oven. Thereby, the material is overflowed with 6 l/min air. In Table 6, the exemplary results are summarized for the reaction of butadiene to vinyl oxirane by using the catalyst T2530. The testing is carried out analogously to Example 1. It is shown here, as in the last Example, that conversions, selectivities and yields are achieved, which are in total clearly lower, if the growth of the silver crystals is not controlled by means of avoiding exposing and by means of cooling.

TABLE 6

| catalyst number | mounting space | temperature [° C.] | GHSV [h⁻¹] | conversion | sel. to VO | yield VO |
|---|---|---|---|---|---|---|
| T2530 | 1.0 mL | 220 | 12000 | 0.00 | 0.00 | 0.00 |
| T2530 | 1.0 mL | 240 | 12000 | 7.24 | 100.00 | 7.24 |
| T2530 | 1.0 mL | 260 | 12000 | 24.91 | 100.00 | 24.91 |
| T2530 | 1.0 mL | 280 | 12000 | 41.57 | 100.00 | 41.57 |
| T2530 | 1.0 mL | 300 | 12000 | 80.36 | 38.86 | 31.23 |
| T2530 | 1.0 mL | 320 | 12000 | 100.00 | 0.14 | 0.14 |
| T2530 | 1.0 mL | 340 | 12000 | 100.00 | 0.00 | 0.00 |
| T2530 | 1.0 mL | 340 | 12000 | 100.00 | 0.00 | 0.00 |
| T2530 | 1.0 mL | 360 | 12000 | 100.00 | 0.00 | 0.00 |
| T2530 | 1.0 mL | 380 | 12000 | 100.00 | 0.00 | 0.00 |

1-11. (canceled)

12. Process for the manufacture of a catalyst, which contains at least silver, comprising at least one of the two following steps:
(I) reaction of a silver-containing component with at least one complexing agent,
(II) reaction of a silver-containing component with at least one reducing agent,
wherein at least one step is carried out at least partially in the absence of light or at temperatures below the room temperature or in absence of light and at temperatures below the room temperature.

13. Process according to claim 12, wherein the process optionally contains at least one further of the following steps:
(III) bringing into contact of the silver-containing component from (I) and/or (II) with a support,
(IV) calcination of the support from (III), which was brought into contact,
wherein one of said steps, or both steps, optionally can be carried out in absence of light or at temperatures below the room temperature, or in absence of light and at temperatures below the room temperature.

14. Process according to claim 13, wherein the step of the calcination takes place at temperatures from 200° C. to below 300° C.

15. Process according to at least one of the preceding claims, wherein the at least one complexing agent is selected from the group containing amines, diamines, alcohols, alkanediols, aminoalcohois, EDTA, carboxylic acids, funtionalized carboxylic acid and carboxylic diacids as well as that the at least one reducing agent is selected from the group containing alkanes, amines, alcohols, hydrogen, hydrogen-containing compounds, aminoalcohols, carboxylic acids.

16. Process according to claim 12 wherein the at least one catalyst is applied on a support as active mass or is brought into contact with a support, wherein the support is selected from the following group containing silicates; alumina oxides; oxides of the metals of the Main and Auxiliary Groups; mixed oxides, or mixed oxides or oxides, in which parts of the lattice sites of a pure oxide are replaced by at least one further element; carbon-containing substances; nitrides as well as mixtures of at least two of the before-mentioned support materials.

17. Catalyst, which contains at least silver, produced by a process comprising at least one of the two following steps:
(I) reaction of a silver-containing component with at least one complexing agent,
(II) reaction of a silver-containing component with at least one reducing agent,
as well as that at least one of said steps is carried out at least partially in absence of light or at temperatures below the room temperature or in absence of light and at temperatures below the room temperature.

18. Catalyst according to claim 17, wherein besides silver at least one further element of the Periodic Table of the Elements is present.

19. Catalyst according to claim 18, wherein the at least one further element is selected from the group comprising K, Rb, Cs, Sr and Ba.

20. Use of the catalyst of claim 18 or 19, or the catalyst produced with a process according to at least one of the claims 12-14, for the reaction of at least one substrate with at least one C—C-double bond in presence of at least one oxygen-containing or oxygen-supplying component.

21. Use according to claim 20, wherein the at least one substrate with C—C-double bond is 1,3-butadiene, and that as product at least vinyl oxirane yields, or that the at least one substrate with C—C-doublt bond is 1,3-butadiene or that as product at least vinyl oxirane yields.

22. Use according to claim 20 wherein the reaction takes place in a fixed bed.
